(19) United States
(12) Patent Application Publication
(10) Pub. No.: US 2017/0087186 A1
(43) Pub. Date: Mar. 30, 2017

(54) BIO-ACTIVE PEPTIDE SUPPLEMENT

(71) Applicant: iSatori, Inc., Golden, CO (US)

(72) Inventor: Stephen Daniel Adele, Golden, CO (US)

(73) Assignee: iSatori, Inc., Golden, CO (US)

(21) Appl. No.: 15/231,879

(22) Filed: Aug. 9, 2016

Related U.S. Application Data
(63) Continuation of application No. 14/299,664, filed on Jun. 9, 2014, now abandoned.
(60) Provisional application No. 61/832,610, filed on Jun. 7, 2013.

(51) Int. Cl. A61K 35/20; A61K 38/40; A61K 38/30; A61K 39/395; A61K 38/18

(52) U.S. Cl. CPC A61K 35/20; A61K 39/39508; A61K 38/1808; A61K 38/1825; A61K 38/1841; A61K 38/1858; A61K 38/30; A61K 38/40

(57) ABSTRACT

A colostrum derived bioactive supplement derived of a number of cleaved protein fragments. The colostrum supplement is utilized with milk protien and Leucine to provide improved muscular function and exercise recovery.

### CROSS-REFERENCE

This application is a continuation application of U.S. patent application Ser. No. 14/299,664, filed Jun. 9, 2014, and claims the benefit of the filing date of U.S. Provisional Patent Application No. 61/832,610 having a filing date of Jun. 7, 2013, the entire contents of both applications are incorporated herein by reference.

### FIELD

The present disclosure is directed to a bio-active supplement. More specifically, the present disclosure is directed to a bio-active peptide supplement derived from mammalian colostrum and its use in resistance training and improving muscular function, strength and recovery.

### BACKGROUND

Individuals who exercise sometimes utilize nutritional supplements to increase skeletal, muscle mass and/or strength and/or for recovery. For this purpose, and to enhance recovery from exercise, a variety of nutritional supplements are available for use immediately before or after strenuous exercise wherein both muscle protein synthesis and breakdown may be increased. Such nutritional supplements purportedly improve physical performance and recovery from such exercise and commonly utilize mixtures containing carbohydrate, creatine, protein, dietarily essential and non-essential amino acids, vitamins and/or minerals.

One potential area of interest for such nutritional supplements are growth factors, which are naturally occurring substances that are capable of stimulating cellular growth and proliferation. Along these lines, colostrum is well known for its content of bioactive components including numerous growth factors and other bioactive substances, including (but not limited to) immunoglobulins, lactoferrin, IGF-1, lysozyme, proline-rich peptides, and glycoproteins. Colostrum is the initial fluid secreted by the mammary gland immediately after parturition and serves to provide nutrition, growth factors and immunity to the neonate. Typically, the concentrations of growth factors in colostrum are considerably in excess of that of normal milk. Accordingly, there have been some attempts to utilize colostrum as a nutritional supplement for promoting muscle growth and recovery.

### SUMMARY OF THE INVENTION

Broadly, one aspect disclosed herein is colostrum derived bioactive supplement which may be produced in a number of forms (e.g., powder, pill, tablet). The colostrum derived supplement is comprised of a number of cleaved protein fragments that are extracted from the first milking colostrum, centrifugated, isolated and concentrated to form a low molecular weight, highly bioavailable, exact bioactive concentrate. The source of colostrum may be from any mammal. Preferably the mammal will be selected from the group including (but not limited to) the bovine, ovine, porcine, caprine or equine. More preferably, the colostrum is from the bovine.

In general, a number of growth factors are isolated from colostrum and concentrated to provide a physiological effect that, once consumed, signals the body to amplify its natural muscle protein synthesis process, helping users develop lean muscle and recover faster from intense workouts. The supplement does this by assisting a user's body to signal and accelerate the rate at which it processes the building and re-building of muscle tissue, called muscle protein synthesis. By doing so, a user can develop lean body mass at a faster rate, more efficiently. Additionally, a user will notice a reduction in recovery time after bouts of intense exercise or weight-training.

In one aspect, the supplement utilizes at least three bioactive components isolated from colostrum and fortified with Leucine, an essential amino acid. More specifically, the supplement in a base form includes IGF-1, transforming growth factor-beta 2 (TGF-β2), and proline rich peptides (PRPs). Though colostrum has some base levels of Leucine, these levels are believed to be too low to produce efficient muscle protein synthesis. Accordingly, additional Leucine is added to the bioactive peptide supplement. In various arrangements, the individual concentrations of the bioactive components may be varied. For instance, the IGF-1 may be in a concentration of at least 0.6 mcg/g of supplement; TGF-β2 may be in a concentration of at least 0.7 mcg/g of the supplement; the PRPs may be in a concentration of at least 15 mg/g of the supplement and more preferably at least 30 mg/g. Preferably the concentrations of the growth factors, such as IGF-1, TGF-β2 and PRP, are at least equal to or excess of the concentrations found in normal unprocessed colostrum. Leucine is typically at least in twice the concentration found in normal unprocessed colostrum, on a gram per gram basis. Further, the isolation of these components from higher abundance proteins in the colostrum proteome is believed to increase their effectiveness in a supplement.

The inclusion of IGF-1 and TGF-β2 enhance the body's protein synthesis. That is, these growth factors promote physiological response to distress signals generated by the body in response to muscle exertion and thereby improve overall protein synthesis. More specifically, IGF-1 is a primary mediator of the effects of growth hormone (GH) made in anterior pituitary gland, which is released into the blood stream and then stimulates systemic body growth. IGF-1 has been identified as having growth-promoting effects on almost every cell in the body, especially skeletal muscle, cartilage, bone, liver, kidney, nerves, skin, hematopoietic cell, and lungs. Transforming growth factor-beta 2 (TGF-β2) are multifunctional peptides that regulate proliferation, differentiation, adhesion, migration, and other functions in many cell types by transducing their signal through cell membranes. PRPs have the ability to modulate the immune system, increasing its activity level in the case of a challenge, such as muscle exertions. When cell damage is detected, chemical signals go out to mobilize other cells to begin repair. PRPs are one of the primary signals that go out at this time. Stated otherwise, PRPs are cell-signalers that, within the muscle cell, initiate the call for amino acids, to induce protein synthesis. Which in turn, helps the muscle cell to undergo more efficient muscle protein synthesis. By increasing the PRPs available to the body, the signal level generated by the body may be amplified, thereby improving recovery.

In addition to the above noted bioactive components, the supplement may include additional components, some of which may likewise be isolated and/or concentrated from colostrum. In one arrangement, the supplement further includes Immunoglobulin A (IgA), which is an antibody that plays a critical role in mucosal immunity. Stated otherwise, IgA is an anti-inflammatory that assists in the recovery of white blood cells. In one arrangement, IgA may be present in a concentration of at least 3 mg/g and 15 mg/g of said supplement. In a further arrangement, Immunoglobulin G (IgG), may be included in the supplement. IgG is an antibody isotype that controls infection of body tissues. IgG has an antibiotic effect, which in turn, helps the muscle cell and neuromuscular connection to become more efficient and “fire” with more precision. IgG may be present in a concentration of between 200 mg/g and 500 mg/g of the supplement. In a yet further arrangement, Lactoferrin (LF) may be included in the supplement. LF, like IgA, stimulates the immune system. More importantly, LF has a high infinity to bind to iron. Which in turn, helps the muscle cell to rebuild and recover more efficiently. LF may be present in at least a concentration of 2 mg/g of said supplement. IgA and LF may each be isolated from colostrum.

In further arrangements, additional bioactive components may be present in the supplement. Such components include, Fibroblast growth factors (FGFs), which are a family of growth factors involved in angiogenesis, wound healing, and embryonic development; Epidermal growth factor (EGF) which is a growth factor that stimulates cell growth, proliferation, and differentiation; and/or Platelet-derived growth factor (PDGF), which regulate cell growth and division.

The above-noted supplement in any combination may be further incorporated into and/or utilized with additional components. In one arrangement, the colostrum derived supplement is utilized with whey protein. In such an arrangement, the whey protein may constitute a majority of the resulting supplement. In a further arrangement, Leucine may also be incorporated in such a combined supplement.

In one arrangement, the supplement is in the form of a powder that may mixed with a liquid base for ingestion. Due to the concentration of the specific bioactive components, the resulting supplement may be 25 times more concentrated than a regular whey protein supplement. In this regard, a 1.5 gram scoop of the bioactive supplement equals the bioactives of 25 grams of a whey protein concentrate. The reduced volume of the supplement facilitates use by individuals.

In another aspect, the bioactive supplement is utilized (e.g., ingested daily) in conjunction with a training regimen to produce one or more enhanced physiological effects. In this regard, the bioactive supplement/product and training has been found to increase the following physiological responses:

Bioactive Product+training resulted in greater muscle hypertrophy than training alone.

Bioactive Product+training resulted in greater muscle strength and power than training alone.

Bioactive Product+training resulted in greater neuromuscular efficiency changes than training alone−less muscle to do more work.

Bioactive Product+training resulted in greater skeletal muscle physiological cross-sectional area than training alone.

Bioactive Product+training resulted in greater muscle quality changes than training alone.

Ingestion of Bioactive Product with Leucine and or Whey protein enhances the effectiveness of muscle growth greater than bioactive product ingested only.

Ingestion of Bioactive Product with Leucine and or Whey protein enhances muscle quality more than bioactive product ingested only.

### DETAILED DESCRIPTION

Colostrum is the pre-milk produced immediately after birth before the breast secretions stabilize into milk. Colostrum includes concentrations of immunoglobulins, cytokines, interferons, glycoproteins, proline-rich peptides and vitamins which are significantly higher than in regular milk. In addition colostrum also includes various growth factors (e.g. IGF-I, TGF A and B), which are likewise in concentrations significantly higher than regular milk The immunoglobulin fraction of the colostrum provides a newborn with antibodies, lactoferrin and immune enhancers. These components offer the newborn protection against viruses, bacteria, allergens and toxins. The growth factors enable the newborn to grow healthily and to heal rapidly. That even healthy adults may benefit from the administration of colostrum is suggested by studies showing that bovine colostrum whey increases serum levels of insulin-like growth factor (IGF-I) in athletes undergoing training: Mero et al., 1997, J. Appl. Physiol., 1997, 83: 1144-1151. In sum, colostrum contains powerful healing, growth and repair factors that activate numerous immune, healing, growth and repair systems and assist in synthesis, retention and repair of muscle, bone, nerve and cartilage. Accordingly, it is desirable to concentrate one or more of the components of colostrum to provide a nutritional supplement for exercise recovery.

Colostrum components of interest include immunoglobulins which have antibiotic effects. Within muscle cells IGs are scavengers for detecting harmful substances, which in turn, helps the muscle cell and neuromuscular connection to become more efficient and “fire” with more precision. PRPs (Proline Rich Peptides) are cell-signalers that, within the muscle cell, initiate the call for amino acids, to induce protein synthesis. Which in turn, helps the muscle cell to undergo more efficient muscle protein synthesis. Lactoferrin has beneficial effects on “good” bacteria and immunity, but also because it has a high infinity to bind to iron. Which in turn, helps the muscle cell to rebuild and recover more efficiently. IGF-1 is a primary mediator of the effects of growth hormone (GH) made in anterior pituitary gland, which is released into the blood stream and then stimulates systemic body growth. IGF-1 has been identified as having growth-promoting effects on almost every cell in the body, especially skeletal muscle, cartilage, bone, liver, kidney, nerves, skin, hematopoietic cell, and lungs. Transforming growth factor-beta 2 (TGF-β2) are multifunctional peptides that regulate proliferation, differentiation, adhesion, migration, and other functions in many cell types by transducing their signal through cell membranes. Generally, these components, among others, are considered bio-active peptides, which are defined as protein fragments that have a positive impact on body functions

A colostrum supplement or extract including various ones, all of the above-noted bio-active peptides and/or additional peptides enhance a body's ability to recover from a workout. However, it is believed that taken alone, such a colostrum extract may not achieve maximum effectiveness. That is, while the bio-active peptide supplement may provide various growth factors and/or amino acids (e.g., building blocks), the body needs additional inputs to efficiently recover.

The addition of whey protein to the human diet has been proven to be a successful adjunct to regular meals for both health and fitness related parameters. It is well known that Milk Derived Proteins including but not limited to whey protein in concentrate, isolate, hydrolysate, and casein protein forms have been shown to improve muscle size, strength and definition (tone). Protein supplementation has been shown to have a positive effect on the immune, cardiovascular, nervous, and gastrointestinal systems through various mechanisms as the proteins are broken down into their active constituents and absorbed. Further evidence suggests that bio-active peptides found within whey protein may have an even greater effect on those individual systems as well as improving hormone levels that play a specific role in musculoskeletal maintenance.

From an anabolic standpoint, whey protein is comprised of several amino acids and other constituents that aid in muscle protein synthesis, specific metabolic functions, and autoregulation of various systems while both at rest and exercising. Several studies have shown that increased protein consumption along with training has improved recovery time, increased muscle mass and improved Muscle Protein Synthesis. Protein-based supplement consumption has been shown to increase insulin secretion which in addition to its role in glucose metabolism, it plays a key role in the process of Muscle Protein Synthesis. Additionally, protein supplementation has been shown to help with satiety, curbing appetite, while providing key nutrients to the body, thus making it a valuable adjunct for helping maintain lean muscle tissue while persons are engaged in a calorie-restricted diet.

Of the 20 amino acids, 9 are essential, meaning they cannot be derived on their own and must be consumed by food or supplement. The others can be synthesized by the body in a variety of ways and thus are considered non-essential. All the essential aminos are needed to carry out specific bodily functions, but a few play a critical role in Muscle Protein Synthesis (MPS). These muscle-building specialists are collectively known as the Branch-Chained Aminos Acids or BCAAs for short and are individually called Leucine, Iso-Leucine and Valine. They have been given the classification due to their branched structure and shape as compared to other non-branched amino acid strands. The BCAAs, and in particular, Leucine, appears to be the most beneficial amino acid for muscle protein synthesis. Leucine appears to trigger various functions in the muscle building pathway, that force a series of steps to take place which ultimately cause the rebuilding of new protein strands. This process, while not completely understood is downstream from main cellular activity and is perpetuated by the activation of a conduit known as the mTOR Pathway (mammalian target of rapamycin). While the science is complex, increasing mTOR activation means increased protein synthesis. The other Essential Amino Acids play vital roles in hormone regulation, metabolism of various fuels, repair of specific tissues, and central and peripheral nervous system maintenance. All of these factors that are controlled by EAAs, are vital in aiding in recovery, helping with muscle development, and improving physical and athletic performance variables.

To maximize athletic performance and recovery, and in particular, muscle-building capability, muscle protein synthesis needs to be increased and protein degradation needs to be minimized. If the anabolic effect of protein synthesis exceeds that of the catabolic effect of protein breakdown, a positive result occurs for both athletic performance and muscle-building. Since several amino acids and bio-active peptides are responsible for muscle protein synthesis, a higher presence of these in the blood facilitates faster protein synthesis attributing to greater athletic gains. Further combining amino acids, proteins, and bioactive peptides ensures that all pathways and mechanisms of action for improving muscle quality, muscle strength, muscle size, muscle power, muscle endurance and reducing recovery time are greatly enhanced. Bioactive peptides in combination with any single component (protein or amino acid) or in combination with several components, improve all aspects of athletic performance and in particular, muscle development at a considerably greater rate than Amino Acids or Whey Protein on their own. Thus, the addition of Bioactive Peptides to functional protein components and amino acids, produces superior improvement in athletic function then the previous best practice of supplementing with either amino acids or protein.

While a bioactive peptide supplement can be used on its own, when combined with whey and/or casein protein and/or amino acids, it will act synergistically to “fortify” overall effectiveness of all of the constituents. Specifically the combination of bio-active peptides with whey, casein protein, amino acids (particularly L-leucine) in a combined/finished product improves the overall effectiveness of the supplement and maximizes the effectiveness of increasing and improving muscle protein synthesis and decreasing muscle protein breakdown. Specifically, the addition of Leucine (i.e., in addition to any amounts existing in the colostrum extract) increases the amount of free leucine to stimulate mTOR activity and also increases the bioavailability of IGF-1 or receptor activation and augmented activity of the Akt/mTOR signaling pathway.

In summary, the combining of amino acids, whey protein, casein protein and bioactive peptides/colostrum extract is based on the following rationale: bioactive peptides/colostrum extract already have a great complement of amino acids, antibodies and PRPs for boosting immune function and reducing inflammation, and a myriad of important growth factors most notably IGF-1. The addition of additional L-leucine will amplify the response in muscle protein synthesis by activating mTOR signaling. Adding additional essential and non-essential amino acids will amplify the response of muscle protein synthesis, reduce recovery time, and provide important immune support to combat the stress of exercise. Adding whey protein will provide additional essential amino acids and yet Leucine which will further amplify muscle protein synthesis activation. Adding casein protein will help sustain the increase in blood leucine levels for several hours, reduce muscle protein degradation, and also help to increase the bioavailability of IGF-1 contained in the bioactive peptides/colostrum extract.

### EXAMPLE 1

### Bioactive Peptide/Colostrum Extract

A mixture was prepared by separating bioactive components from whole colostrum by the removal of fat, sugar and casein to obtain bovine colostral whey, followed by ultra-filtration and concentration with nano-filtration. Resulting components where combined in a concentration of (Insulin-like Growth Factor-1) IGF-1 at 1.2 mcg/g of supplement; (Transforming growth factor-beta 2) TGF-β2 in a concentration of 1.4 mcg/g of the supplement; Protein Rich Peptides (PRPs) in a concentration of at least 30 mg/g of the supplement, Immunoglobulin A (IgA) in a concentration of 22 mg/g of the supplement and Lactoferrin (LF) in a concentration of at least 5.5 mg/g of the supplement with the remainder comprising trace amounts of other components, protein, sugar and fat.

### EXAMPLE 2

Bioactive Peptides/Colostrum Extract with Milk Protein

500 mg to 10 g of bioactive peptides/colostrum extract, per serving in combination with any or all of the following:

1) 5 g to 30 g Milk Derived Proteins including but not limited to:
a. 5 g to 30 g Whey Protein Concentrate
b. 5 g to 30 g Whey Protein Isolate
c. 5 g to 30 g Whey Protein Hydrolysate
d. 5 g to 30 g Casein Protein

2) 1 g to 6 g Leucine

3) 500 mg to 6 g any BCAA

4) 250 mg to 6 g any Essential Amino Acid

5) 250 mg to 6 g any Amino Acid (non-essential)

The foregoing description has been presented for purposes of illustration and description. Furthermore, the description is not intended to limit the inventions and/or aspects of the inventions to the forms disclosed herein. Consequently, variations and modifications commensurate with the above teachings, and skill and knowledge of the relevant art, are within the scope of the presented inventions. The embodiments described hereinabove are further intended to explain best modes known of practicing the inventions and to enable others skilled in the art to utilize the inventions in such, or other embodiments and with various modifications required by the particular application(s) or use(s) of the presented inventions. It is intended that the appended claims be construed to include alternative embodiments to the extent permitted by the prior art.

1-17. (canceled)

18. A method of increasing athletic performance and recovery, lean body mass, and muscle building capability in a subject who is in a muscle-building training regimen, comprising:
ingesting a daily dose of a bioactive peptide nutritional supplement composition effective to increase athletic performance and recovery, lean body mass, and muscle building capability, wherein the nutritional supplement composition is concentrated from colostrum and comprises:
insulin-like growth factor (IGF-1) in a concentration between 0.6 mcg/g and 1.8 mcg/g;
transforming growth factor-beta 2 (TGF-β2) in a concentration between 0.7 mcg/g and 3.65 mcg/g;
proline rich peptides (PRPs) in a concentration between 30 mg/g and 45 mg/g;
immunoglobulin A in a concentration between 3 mg/g and 20 mg/g; and
immunoglobulin G (IgG) in a concentration of at least 300 mg/g.

19. The method of claim 18, wherein the nutritional supplement is ingested in a dose of 500 mg to 10 g.

20. The method of claim 18, wherein the nutritional supplement composition is mixed with a liquid base for ingestion.

21. The method of claim 18, wherein the nutritional supplement composition further comprises lactoferrin (LF), extracted from colostrum, in at least a concentration of between 2 mg/g and 21 mg/g of said nutritional supplement composition.

22. The method of claim 18, wherein the nutritional supplement composition further comprises at least one of a fibroblast growth factor, epidermal growth factor (EGF), and/or platelet-derived growth factor (PDGF).

23. A method of increasing athletic performance and recovery, lean body mass, and muscle building capability in a subject who is in a muscle-building training regimen, comprising:
ingesting a daily dose of a bioactive peptide nutritional supplement composition effective to increase athletic performance and recovery, lean body mass, and muscle building capability, wherein the nutritional supplement composition is concentrated from colostrum and comprises:
insulin-like growth factor (IGF-1) in a concentration between 0.6 mcg/g and 1.8 mcg/g;
transforming growth factor-beta 2 (TGF-β2) in a concentration between 0.7 mcg/g and 3.65 mcg/g;
proline rich peptides (PRPs) in a concentration between 30 mg/g and 45 mg/g;
immunoglobulin A in a concentration between 3 mg/g and 20 mg/g;
immunoglobulin G (IgG) in a concentration of at least 300 mg/g;
lactoferrin (LF), extracted from colostrum, in at least a concentration of between 2 mg/g and 21 mg/g of said nutritional supplement composition;
at least one of:
a fibroblast growth factor;
epidermal growth factor (EGF);
platelet-derived growth factor (PDGF); and
wherein the nutritional supplement is mixed with a liquid base for ingestion and ingested in a dose of 500 mg-10 g.
